(19) United States
(12) Patent Application Publication
(10) Pub. No.: US 2026/0071285 A1
(43) Pub. Date: Mar. 12, 2026

(54) NOVEL QTLS CONFERRING RESISTANCE TO CUCURBIT APHID-BORNE YELLOW VIRUS

(71) Applicant: Seminis Vegetable Seeds, Inc., St. Louis, MO (US)

(72) Inventors: Melissa C. Cantet, Saint Andiol (FR); Severine Monnot, Avignon (FR); Antoon Stekelenburg, Houten (NL)

(21) Appl. No.: 19/324,504

(22) Filed: Sep. 10, 2025

Related U.S. Application Data
(60) Provisional application No. 63/693,543, filed on Sep. 11, 2024.
(60) Provisional application No. 63/710,398, filed on Oct. 22, 2024.

(51) Int. Cl. C12Q 1/6895

(52) U.S. Cl. CPC C12Q 1/6895; C12Q 2600/13; C12Q 2600/156

(57) ABSTRACT

Cucumber plants exhibiting resistance to cucurbit aphid-borne yellows virus (CABYV) are provided, together with methods of producing, identifying, or selecting plants or germplasm with a cucurbit aphid-borne yellows virus resistance phenotype. Such methods include producing a cucumber plant exhibiting resistance to CABYV, comprising introgressing genomic regions conferring disease resistance; or selecting a cucumber plant exhibiting resistance to CABYV. Compositions, including polymorphic markers for detecting plants comprising introgressed disease resistance alleles, are further provided.

### CROSS-REFERENCE TO RELATED APPLICATIONS

This application claims the benefit of U.S. Provisional Patent Application No. 63/693,543, filed Sep. 11, 2024, and U.S. Provisional Patent Application No. 63/710,398, filed Oct. 22, 2024, the entire disclosures of which are incorporated herein by reference.

### INCORPORATION OF SEQUENCE LISTING

A sequence listing containing the file named “SEMB059US_ST26.xml” which is 19,721 bytes (measured in MS-Windows®) and created on Sep. 9, 2025, and comprises 13 sequences, is incorporated herein by reference in its entirety.

### FIELD OF THE INVENTION

The present invention relates to the field of plant breeding and, more specifically, to methods and compositions for producing cucumber plants exhibiting improved resistance to cucurbit aphid-borne yellows virus (CABYV).

### BACKGROUND

Cucurbit aphid-borne yellows virus (CABYV) is a Polerovirus of emerging importance to cucurbit growers worldwide. CABYV is one of the most common cucurbit viruses found in open field cucurbit crops grown in a variety of diverse areas and environments. CABYV infection does not typically affect fruit quality itself but can have a significant detrimental effect on fruit yield. Thus, resistance to CABYV is a particularly important trait for the production of cucurbits, especially cucumbers. Although some CABYV resistance alleles have been identified melon, the mapping and introduction of sustainable resistance to viruses remains one of the main challenges of modern plant breeding, especially in cucumber breeding. Moreover, the globalization of food supply chains favors the spread of new virus strains or species. Therefore, a continuing need exists in the art to identify new resistance alleles conferring increased resistance to CABYV as well as more effective methods of introgressing those resistance alleles into commercial lines to provide new varieties with improved resistance to CABYV infection.

### SUMMARY

In one aspect, provided herein is a method of producing a cucumber plant exhibiting resistance to cucurbit aphid-borne yellows virus (CABYV), comprising introgressing into a plant a CABYV resistance allele within a chromosomal segment flanked in the genome of said plant by marker locus M1 (SEQ ID NO:1) and marker locus M2 (SEQ ID NO:10) on chromosome 4, wherein said introgressed CABYV resistance allele confers to said plant increased resistance to CABYV compared to a plant not comprising said allele. In some embodiments, said introgressing comprises: a) crossing a cucumber plant comprising said chromosomal segment with itself or with a second cucumber plant to produce one or more progeny plants; and b) selecting a progeny plant comprising said chromosomal segment. In other embodiments, a representative sample of seed comprising said chromosomal segment has been deposited under NCMA Accession Number 202409001. In some embodiments, said chromosomal segment comprises a marker locus on chromosome 4 selected from the group consisting of marker locus M1 (SEQ ID NO:1), marker locus M3 (SEQ ID NO:2), marker locus M4 (SEQ ID NO:3), marker locus M5 (SEQ ID NO:4), marker locus M6 (SEQ ID NO:5), marker locus M7 (SEQ ID NO:6), marker locus M8 (SEQ ID NO: 7), marker locus M9 (SEQ ID NO:8), marker locus M10 (SEQ ID NO:9), marker locus M11 (SEQ ID NO:11), marker locus M12 (SEQ ID NO:12), marker locus M13 (SEQ ID NO:13), and marker locus M2 (SEQ ID NO:10). In other embodiments, said introgressing comprises backcrossing, marker-assisted selection, or assaying for said CABYV resistance.

In another aspect, cucumber plants are provided that are obtainable by a method disclosed herein, wherein the plants comprise a cucurbit aphid-borne yellows virus (CABYV) resistance allele within a chromosomal segment flanked in the genome of said plant by marker locus M1 (SEQ ID NO:1) and marker locus M2 (SEQ ID NO: 10) on chromosome 4.

In yet another aspect, methods are provided for selecting a cucumber plant with increased resistance to cucurbit aphid-borne yellows virus (CABYV), comprising: (a) crossing a cucumber plant comprising a CABYV resistance allele with a second cucumber plant to produce a population of progeny plants; and (b) selecting a progeny plant comprising said CABYV resistance allele; wherein selecting said progeny plant comprises detecting a marker locus within or genetically linked to a chromosomal segment flanked in the genome of said plant by marker locus M1 (SEQ ID NO: 1) and marker locus M2 (SEQ ID NO:10) on chromosome 4. In some embodiments, selecting a plant or progeny plant comprises detecting nucleic acids comprising marker locus M1 (SEQ ID NO:1), marker locus M3 (SEQ ID NO:2), marker locus M4 (SEQ ID NO:3), marker locus M5 (SEQ ID NO:4), marker locus M6 (SEQ ID NO:5), marker locus M7 (SEQ ID NO:6), marker locus M8 (SEQ ID NO:7), marker locus M9 (SEQ ID NO:8), marker locus M10 (SEQ ID NO: 9), marker locus M11 (SEQ ID NO:11), marker locus M12 (SEQ ID NO:12), marker locus M13 (SEQ ID NO:13), and marker locus M2 (SEQ ID NO:10). In other embodiments, the progeny plant is an F₂-F₆ progeny plant. In further embodiments, producing said plant or progeny plant comprises backcrossing.

In a further aspect, methods are provided for selecting a cucumber plant exhibiting resistance to cucurbit aphid-borne yellows virus (CABYV), comprising: a) obtaining a population of progeny plants having a parent comprising resistance to CABYV; b) screening said population with at least one nucleic acid marker to detect a polymorphism genetically linked to CABYV resistance; and c) selecting from said population one or more plants comprising a haplotype associated with CABYV resistance, wherein the haplotype comprises a CABYV resistance allele flanked in the genome of said plant by marker locus M1 (SEQ ID NO:1) and marker locus M2 (SEQ ID NO:10) on chromosome 4. In some embodiments, selecting said one or more plants comprises: (a) detecting a marker locus within or genetically linked to said chromosomal segment flanked in the genome of said plant by marker locus M1 (SEQ ID NO:1) and marker locus M2 (SEQ ID NO:10) on chromosome 4; or (b) detecting at least one polymorphism at a locus selected from the group consisting of marker locus M1 (SEQ ID NO:1), marker locus M3 (SEQ ID NO:2), marker locus M4 (SEQ ID NO:3), marker locus M5 (SEQ ID NO:4), marker locus M6 (SEQ ID NO: 5), marker locus M7 (SEQ ID NO:6), marker locus M8 (SEQ ID NO:7), marker locus M9 (SEQ ID NO:8), marker locus M10 (SEQ ID NO:9), marker locus M11 (SEQ ID NO:11), marker locus M12 (SEQ ID NO: 12), marker locus M13 (SEQ ID NO:13), and marker locus M2 (SEQ ID NO: 10). In other embodiments, said plant is an F₂-F₆ progeny plant. In further embodiments, producing said plant or progeny plant comprises backcrossing. In some embodiments, screening said population comprises PCR, single strand conformational polymorphism analysis, denaturing gradient gel electrophoresis, cleavage fragment length polymorphism analysis, TAQMAN assay, and/or DNA sequencing. In yet other embodiments, a representative sample of seed comprising said allele has been deposited under NCMA Accession Number 202409001.

In another aspect, methods are provided for selecting a cucumber plant exhibiting resistance to cucurbit aphid-borne yellows virus (CABYV), comprising: a) screening one or more plants with at least one nucleic acid marker to detect a polymorphism genetically linked to CABYV resistance; and b) selecting one or more plants comprising a haplotype associated with CABYV resistance, wherein the haplotype comprises a CABYV resistance allele flanked in the genome of said plant by marker locus M1 (SEQ ID NO:1) and marker locus M2 (SEQ ID NO:10) on chromosome 4. In some embodiments, the CABYV resistance allele is further defined as located within a chromosomal segment flanked in the genome of said plant by marker locus M1 (SEQ ID NO: 1) and marker locus M2 (SEQ ID NO:10) on chromosome 4. In other embodiments, selecting one or more plants comprises: (a) detecting a marker locus within or genetically linked to a chromosomal segment flanked in the genome of said plant by marker locus M1 (SEQ ID NO:1) and marker locus M2 (SEQ ID NO:10) on chromosome 4; or (b) detecting at least one polymorphism at a locus selected from the group consisting of marker locus M1 (SEQ ID NO:1), marker locus M3 (SEQ ID NO:2), marker locus M4 (SEQ ID NO:3), marker locus M5 (SEQ ID NO: 4), marker locus M6 (SEQ ID NO:5), marker locus M7 (SEQ ID NO:6), marker locus M8 (SEQ ID NO:7), marker locus M9 (SEQ ID NO:8), marker locus M10 (SEQ ID NO:9), marker locus M11 (SEQ ID NO:11), marker locus M12 (SEQ ID NO:12), marker locus M13 (SEQ ID NO: 13), and marker locus M2 (SEQ ID NO:10). In further embodiments, screening one or more plants comprises PCR, single strand conformational polymorphism analysis, denaturing gradient gel electrophoresis, cleavage fragment length polymorphism analysis, TAQMAN assay, and/or DNA sequencing.

In yet another aspect, methods are provided herein for identifying a cucumber plant comprising a cucurbit aphid-borne yellows virus (CABYV) resistance allele comprising: a) obtaining nucleic acids from at least a first cucumber plant; and b) identifying in said nucleic acids the presence of at least a first genetic marker indicative of the presence of a chromosomal segment flanked in the genome of said plant by marker locus M1 (SEQ ID NO:1) and marker locus M2 (SEQ ID NO:10) on chromosome 4, wherein said CABYV resistance allele confers to said plant increased resistance to CABYV compared to a plant not comprising said allele. In some embodiments, said identifying comprises detecting a marker genetically linked to marker locus M1 (SEQ ID NO:1), marker locus M3 (SEQ ID NO:2), marker locus M4 (SEQ ID NO:3), marker locus M5 (SEQ ID NO:4), marker locus M6 (SEQ ID NO:5), marker locus M7 (SEQ ID NO:6), marker locus M8 (SEQ ID NO:7), marker locus M9 (SEQ ID NO:8), marker locus M10 (SEQ ID NO: 9), marker locus M11 (SEQ ID NO:11), marker locus M12 (SEQ ID NO:12), marker locus M13 (SEQ ID NO:13), and marker locus M2 (SEQ ID NO:10).

### BRIEF DESCRIPTION OF THE DRAWINGS

FIG. 1: Shows QTL mapping for the CABYV maximum infection rate in open field conditions (OPF). Dots refer to MLMM analyses and bars are for local score analyses. The triangles represent the top SNP of each QTL and its position. The dashed line represents significance (for the MLMM Bonferroni threshold according to the number of independent SNPs calculated by Gao et al. (2011) and for the Lindley process the threshold correcting the genomic inflation per chromosome).

FIG. 2: Shows genetic organization of the hotspot on chromosome 4 in relation with the genetic structure and level of resistance. FIG. 2A shows a heatmap of the local kinship matrix obtained by the VanRaden method (2008) based on 10,233 SNPs located between 8,682,572 bp and 10,599,858 bp on chromosome 4. FIG. 2B shows genetic group of the individuals tested for resistance to CABYV. FIG. 2C shows resistance level of the individual tested for resistance to CABYV. FIG. 2D shows a visualization of the genetic sequence on chromosome 4, where fill color represents the allelic state of each individual for these SNPs.

FIG. 3: Shows genetic organization of the hotspot on chromosome 4 in relation with the genetic structure and level of resistance. FIG. 3A shows a heatmap of the local kinship matrix obtained by the VanRaden method (2008) based on 10,233 SNPs located between 8,682,572 bp and 10,599,858 bp on chromosome 4. FIG. 3B shows a dendrogram representing the clustering analysis, the dot color represents the attribution of each individual to a haplotype for the QTL on chromosome 4. FIG. 3C shows boxplots that represent the variability of CABYV resistance within and across each haplotype.

### DETAILED DESCRIPTION

Cucurbit aphid-borne yellows (CABY) is a disease caused by cucurbit aphid-borne yellows virus (CABYV). This disease is a substantial problem for cucumber producers and is, in general, an important threat against cucurbit crops around the world. Incidences of CABYV are becoming more frequent in areas where its presence was reported earlier, such as the Mediterranean basin and parts of Asia, however there are reports that it has been introduced into new growing areas, such as Northern Europe as well as North and South America. The impact of CABYV infections on yield depends on the host species and cultivar but its effect is especially devastating on cucumber crops. CABYV does not typically affect the quality of the fruit itself but infection by the virus can lead to flower abortion. The reduction of flowers on a plant directly results in a reduced number of fruit produced on each plant. In particular, CABYV infection is especially devastating in melon and cucumber, as a significant (as much as 50%) reduction in fruit number per plant was observed in infected plants.

Protection of crops from CABYV is based on chemical control of its aphid vectors, however this approach has the disadvantages of potential damage to the environment and risk of selection of resistant aphid populations. Physical barriers against the virus-carrying aphids, such as plastic covers during the early stages of plant development, have been found to result in a delay in the establishment of infections, but does not confer permanent or efficient protection. Under these circumstances, the use of cultivars that carry genetic resistance to the virus is the most desirable form of disease control. A small number of sources for CABYV resistance have been identified in melon however there are no reports of CABYV resistance found in cucumber.

The present disclosure identifies, for the first time, a locus in cucumber that confers intermediate resistance to CABYV. This locus was identified in a region spanning from 8,682,572 bp to 10,505,104 bp on chromosome 4 of the public cucumber genome map version Cucumber Chinese Long′ (CCL) landrace, v3 (Li et al., 2019).

As disclosed herein, M1, a SNP marker with a [A/G] change at 8,682,572 bp on chromosome 4 of the public cucumber genome map version Cucumber Chinese Long′ (CCL) landrace, M3, a SNP marker with a [A/G] change at 8,872,385 bp on chromosome 4 of the public cucumber genome map version 3 Cucumber Chinese Long′ (CCL) landrace, and M4, a SNP marker with a [G/C] change at 9,042,752 bp on chromosome 4 of the public cucumber genome map version Cucumber Chinese Long′ (CCL) landrace, M5, a SNP marker with a [G/T] change at 9,310,448 bp on chromosome 4 of the public cucumber genome map version 3 Cucumber Chinese Long′ (CCL) landrace, and M6, a SNP marker with a [C/T] change at 9,561,517 bp on chromosome 4 of the public cucumber genome map version 3 Cucumber Chinese Long′ (CCL) landrace, and M7, a SNP marker with a [A/G] change at 9,734,323 bp on chromosome 4 of the public cucumber genome map version 3 Cucumber Chinese Long′ (CCL) landrace, and M8, a SNP marker with a [C/T] change at 9,898,582 bp on chromosome 4 of the public cucumber genome map version 3 Cucumber Chinese Long′ (CCL) landrace, and M9, a SNP marker with a [T/C] change at 10,100,790 bp on chromosome 4 of the public cucumber genome map version 3 Cucumber Chinese Long′ (CCL) landrace, and M10, a SNP marker with a [A/T] change at 10,363,190 bp on chromosome 4 of the public cucumber genome map version 3 Cucumber Chinese Long′ (CCL) landrace, and M11, a SNP marker with a [T/C] change at 9,876,485 bp on chromosome 4 of the public cucumber genome map version 3 Cucumber Chinese Long′ (CCL) landrace, and M12, a SNP marker with a [C/T] change at 9,744,356 bp on chromosome 4 of the public cucumber genome map version 3 Cucumber Chinese Long′ (CCL) landrace, and M13, a SNP marker with a [A/G] change at 9,876,485 bp on chromosome 4 of the public cucumber genome map version 3 Cucumber Chinese Long′ (CCL) landrace, and M2, a SNP marker with a [A/G] change at 10,505,104 bp on chromosome 4 of the public cucumber genome map version 3 Cucumber Chinese Long′ (CCL) landrace can be used to identify this region, wherein M1 and M2 or M11 and M13 are flanking markers for the chromosomal segment on chromosome 4. The public genome of cucumber is available at, for example, Cucumber (Chinese Long) genome v3 at CuGenDB (http://cucurbitgenomics.org/) or at the Genbank assembly accession GCA_000004075.3 from NCBI (https://www.ncbi.nlm.nih.gov/genome/1639?genome_assembly_id=749658), and one skilled in the art would understand that the marker sequences provided for the first time in association with CABYV resistance in the instant application could be located on any version (or later version) of the public genome. One aspect of the present disclosure therefore provides methods of producing a cucumber plant exhibiting resistance to cucurbit aphid-borne yellows virus (CABYV), comprising introgressing into a plant a cucurbit aphid-borne yellows virus resistance allele within a chromosomal segment flanked in the genome of the plant by marker locus M1 (SEQ ID NO:1) and marker locus M2 (SEQ ID NO:10) on chromosome 4; wherein the introgressed CABYV resistance allele confers to the plant increased resistance to CABYV compared to a plant not comprising the allele.

In another aspect, cucumber plants are provided that are obtainable by a method disclosed herein, wherein the plants comprise the CABYV resistance allele within a chromosomal segment flanked in the genome of the plant by marker locus M1 (SEQ ID NO:1) and marker locus M2 (SEQ ID NO:10) on chromosome 4. In particular embodiments, said plants are not exclusively obtained by means of an essentially biological process.

In some embodiments, provided herein are methods of selecting a cucumber plant exhibiting resistance to cucurbit aphid-borne yellows virus (CABYV). In certain embodiments, said methods comprise screening one or more plants with at least one nucleic acid marker to detect a polymorphism genetically linked to CABYV resistance, and selecting one or more plants comprising said polymorphism genetically linked to CABYV resistance. In some embodiments, said selecting comprises detecting a marker locus within or genetically linked to a chromosomal segment flanked in the genome of said plant by marker locus M1 (SEQ ID NO: 1) and marker locus M2 (SEQ ID NO:10) on chromosome 4. In other embodiments, said selecting comprises detecting a marker locus within or genetically linked to a chromosomal segment flanked in the genome of said plant by marker locus M11 (SEQ ID NO:11) and marker locus M13 (SEQ ID NO:13) on chromosome 4. In other embodiments, said methods comprise: a) screening one or more plants with at least one nucleic acid marker to detect a polymorphism genetically linked to CABYV resistance; and b) selecting one or more plants comprising a haplotype associated with CABYV resistance, wherein the haplotype comprises a CABYV resistance allele flanked in the genome of the plant by marker locus M1 (SEQ ID NO:1) and marker locus M2 (SEQ ID NO:10) on chromosome 4. In particular embodiments, selecting a cucumber plant exhibiting resistance to cucurbit aphid-borne yellows virus (CABYV) comprises molecular genetic techniques. For example, those of ordinary skill in the art viewing the present disclosure may use technical methods to select a cucumber plant exhibiting resistance to cucurbit aphid-borne yellows virus (CABYV) by screening one or more plants with at least one nucleic acid marker to detect a polymorphism genetically linked to CABYV resistance.

In some embodiments, the present disclosure provides the markers shown in Tables 4 and 5, which have been shown to be genetically linked to cucurbit aphid-borne yellows virus resistance in plants.

In particular embodiments, plants comprising the CABYV resistance alleles are provided. The CABYV resistance alleles described herein provide robust resistance to cucurbit aphid-borne yellows virus. Methods of producing the plants described herein are further provided. The disclosure further provides trait-linked markers which can be used to produce and/or select plants comprising the CABYV resistance allele on chromosome 4 conferring cucurbit aphid-borne yellows virus resistance as described herein.

The present disclosure provides significant advancements in obtaining CABYV resistance in cucumbers by identifying a QTL on chromosome 4. This QTL is distinct from those known in the art. In addition, markers associated with the resistance allele is provided, allowing the allele to be accurately introgressed and tracked during plant breeding. As such, the present disclosure permits introgression of the disease resistance allele into any desired cucumber genotype.

### I. GENOMIC REGIONS, ALLELES, AND POLYMORPHISMS ASSOCIATED WITH CUCURBIT APHID-BORNE YELLOWS VIRUS RESISTANCE IN CUCUMBER PLANTS

Provided herein are introgressions of one or more alleles associated with cucurbit aphid-borne yellows virus resistance, together with polymorphic nucleic acids and linked markers for tracking the introgressions during plant breeding.

Using the improved genetic markers and the assays described herein, those skilled in the art are able to successfully produce and/or select plants comprising the cucurbit aphid-borne yellows virus resistance alleles described herein, which confer increased resistance to CABYV as compared to a plant not comprising the allele(s). In certain embodiments, provided herein are methods of introgressing into a plant a cucurbit aphid-borne yellows virus resistance allele within a chromosomal segment flanked in the genome of the plant by marker locus M1 (SEQ ID NO:1) and marker locus M2 (SEQ ID NO:10) on chromosome 4. The present disclosure therefore represents a significant advance in the art.

### II. INTROGRESSION OF GENOMIC REGIONS ASSOCIATED WITH CUCURBIT APHID-BORNE YELLOWS VIRUS RESISTANCE

Marker-assisted introgression involves the transfer of a chromosomal region defined by one or more markers from a first genetic background to a second. Offspring of a cross that contain the introgressed genomic region can be identified by the combination of markers characteristic of the desired introgressed genomic region from a first genetic background and both linked and unlinked markers characteristic of the second genetic background.

Provided herein are accurate markers for identifying and tracking introgression of one or more of the genomic regions disclosed herein from a CABYV resistant plant into a cultivated line. Further provided are markers for identifying and tracking the introgressions disclosed herein during plant breeding, including the markers set forth in Tables 4 and 5. Markers within or linked to any of the genomic intervals described herein may be useful in a variety of breeding efforts that include introgression of genomic regions associated with disease resistance into a desired genetic background. For example, a marker within 40 cM, 20 cM, 15 cM, 10 cM, 5 CM, 2 cM, or 1 cM of a marker associated with disease resistance described herein can be used for marker-assisted introgression of genomic regions associated with a disease resistant phenotype.

Methods of producing, selecting, and identifying cucumber plants comprising one or more introgressed regions associated with a desired phenotype wherein at least 10%, 25%, 50%, 75%, 90%, or 99% of the remaining genomic sequences carry markers characteristic of the recurrent parent germplasm are also provided. Methods of producing, selecting, and identifying cucumber plants comprising an introgressed region comprising regions closely linked to or adjacent to the genomic regions and markers provided herein and associated with a disease resistance phenotype are also provided.

### III. DEVELOPMENT OF DISEASE RESISTANT CUCUMBER VARIETIES

For most breeding objectives, commercial breeders work within germplasm that is “cultivated,” “cultivated type,” or “elite.” These cultivated lines may be used as recurrent parents or as a source of recurrent parent alleles during breeding. Cultivated or elite germplasm is easier to breed because it generally performs well when evaluated for horticultural performance. Many cultivated cucumber types have been developed and are known in the art as being agronomically elite and appropriate for commercial cultivation. However, the performance advantage a cultivated germplasm provides can be offset by a lack of allelic diversity. Breeders generally accept this tradeoff because progress is faster when working with cultivated material than when breeding with genetically diverse sources.

In contrast, when cultivated germplasm is crossed with non-cultivated germplasm, a breeder can gain access to novel alleles from the non-cultivated type. Non-cultivated germplasm may be used as a source of donor alleles during breeding. However, this approach generally presents significant difficulties due to fertility problems associated with crosses between diverse lines, and negative linkage drag from the non-cultivated parent. For example, non-cultivated cucumber types can provide alleles associated with disease resistance. However, these non-cultivated types may have poor horticultural qualities such as poor fruit shape, agronomically unacceptable plant architecture, and/or necrosis.

The process of introgressing desirable resistance genes from non-cultivated lines into elite cultivated lines while avoiding problems with linkage drag or low heritability is a long and often arduous process. In deploying alleles derived from wild relatives it is often desirable to introduce a minimal or truncated introgression that provides the desired trait but lacks detrimental effects. To aid introgression reliable marker assays are preferable to phenotypic screens. Success is furthered by simplifying genetics for key attributes to allow focus on genetic gain for quantitative traits such as disease resistance. Moreover, the process of introgressing genomic regions from non-cultivated lines can be greatly facilitated by the availability of accurate markers for MAS.

One of skill in the art would therefore understand that the alleles, polymorphisms, and markers provided by the present disclosure allow the tracking and introduction of any of the genomic regions identified herein into any genetic background. In addition, the genomic regions associated with disease resistance disclosed herein can be introgressed from one genotype to another and tracked using MAS. Thus, the disclosure of accurate markers associated with disease resistance will facilitate the development of cucumber plants having beneficial phenotypes. For example, seed can be genotyped using the markers of the present disclosure to select for plants comprising desired genomic regions associated with disease resistance. Moreover, MAS allows identification of plants homozygous or heterozygous for a desired introgression.

Inter-species crosses can also result in suppressed recombination and plants with low fertility or fecundity. For example, suppressed recombination has been observed for the tomato nematode resistance gene Mi, the Mla and Mlg genes in barley, the Yr17 and Lr20 genes in wheat, the Run1 gene in grapevine, and the Rma gene in peanut. Meiotic recombination is essential for classical breeding because it enables the transfer of favorable alleles across genetic backgrounds, the removal of deleterious genomic fragments, and pyramiding traits that are genetically tightly linked. Therefore, suppressed recombination forces breeders to enlarge segregating populations for progeny screens in order to arrive at the desired genetic combination.

Phenotypic evaluation of large populations is time-consuming, resource-intensive and not reproducible in every environment. Marker-assisted selection offers a feasible alternative. Molecular assays designed to detect unique polymorphisms, such as SNPs, are versatile. However, they may fail to discriminate alleles within and among cucumber species in a single assay. Structural rearrangements of chromosomes such as deletions impair hybridization and extension of synthetically labeled oligonucleotides. In the case of duplication events, multiple copies are amplified in a single reaction without distinction. The development and validation of accurate and highly predictive markers are therefore essential for successful MAS breeding programs.

### IV. MARKER-ASSISTED BREEDING AND GENETIC ENGINEERING TECHNIQUES

Genetic markers that can be used in the practice of the present invention include, but are not limited to, restriction fragment length polymorphisms (RFLPs), amplified fragment length polymorphisms (AFLPs), simple sequence repeats (SSRs), simple sequence length polymorphisms (SSLPs), single nucleotide polymorphisms (SNPs), insertion/deletion polymorphisms (Indels), variable number tandem repeats (VNTRs), and random amplified polymorphic DNA (RAPD), isozymes, and other markers known to those skilled in the art. Marker discovery and development in crop plants provides the initial framework for applications to marker-assisted breeding activities (U.S. Patent Pub. Nos.: 2005/0204780, 2005/0216545, 2005/0218305, and 2006/00504538). The resulting “genetic map” is the representation of the relative position of characterized loci (polymorphic nucleic acid markers or any other locus for which alleles can be identified) to each other.

Polymorphisms comprising as little as a single nucleotide change can be assayed in a number of ways. For example, detection can be made by electrophoretic techniques including a single strand conformational polymorphism (Orita et al., Genomics 8(2):271-278, 1989), denaturing gradient gel electrophoresis (Myers (1985) EPO 0273085), or cleavage fragment length polymorphisms (Life Technologies, Inc., Gaithersburg, MD), but the widespread availability of DNA sequencing often makes it easier to simply sequence amplified products directly. Once the polymorphic sequence difference is known, rapid assays can be designed for progeny testing, typically involving some version of PCR amplification of specific alleles (PASA; Sommer et al., Biotechniques 12(1):82-87, 1992), or PCR amplification of multiple specific alleles (PAMSA; Dutton and Sommer, Biotechniques, 11(6):700-7002, 1991).

Polymorphic markers serve as useful tools for assaying plants for determining the degree of identity of lines or varieties (U.S. Pat. No. 6,207,367). These markers form the basis for determining associations with phenotypes and can be used to drive genetic gain. In certain embodiments, polymorphic nucleic acids can be used to detect in a cucumber plant a genotype associated with disease resistance, identify a cucumber plant with a genotype associated with disease resistance, and to select a cucumber plant with a genotype associated with disease resistance. In certain embodiments of methods described, polymorphic nucleic acids can be used to produce a cucumber plant that comprises in its genome an introgressed locus associated with disease resistance. In certain embodiments, polymorphic nucleic acids can be used to breed progeny cucumber plants comprising a locus or loci associated with disease resistance.

Genetic markers may include “dominant” or “codominant” markers. “Codominant” markers reveal the presence of two or more alleles (two per diploid individual). “Dominant” markers reveal the presence of only a single allele. Markers are preferably inherited in codominant fashion so that the presence of both alleles at a diploid locus, or multiple alleles in triploid or tetraploid loci, are readily detectable, and they are free of environmental variation, i.e., their heritability is 1. A marker genotype typically comprises two marker alleles at each locus in a diploid organism. The marker allelic composition of each locus can be either homozygous or heterozygous. Homozygosity is a condition where both alleles at a locus are characterized by the same nucleotide sequence. Heterozygosity refers to a condition where the two alleles at a locus are different.

Nucleic acid-based analyses for determining the presence or absence of the genetic polymorphism (i.e. for genotyping) can be used in breeding programs for identification, selection, introgression, and the like. A wide variety of genetic markers for the analysis of genetic polymorphisms are available and known to those of skill in the art. The analysis may be used to select for genes, portions of genes, QTL, alleles, or genomic regions that comprise or are linked to a genetic marker that is linked to or associated with disease resistance in cucumber plants.

As used herein, nucleic acid analysis methods include, but are not limited to, PCR-based detection methods (for example, TaqMan assays), microarray methods, mass spectrometry-based methods and/or nucleic acid sequencing methods, including whole genome sequencing. In certain embodiments, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

One method of achieving such amplification employs the polymerase chain reaction (PCR) (Mullis et al. Cold Spring Harbor Symp. Quant. Biol. 51:263-273, 1986; European Patent 50,424; European Patent 84,796; European Patent 258,017; European Patent 237,362; European Patent 201,184; U.S. Pat. Nos. 4,683,202; 4,582,788; and 4,683,194), using primer pairs that are capable of hybridizing to the proximal sequences that define a polymorphism in its double-stranded form. Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981; and 7,250,252, all of which are incorporated herein by reference in their entirety. However, the compositions and methods of the present disclosure can be used in conjunction with any polymorphism typing method to detect polymorphisms in genomic DNA samples. These genomic DNA samples used include but are not limited to, genomic DNA isolated directly from a plant, cloned genomic DNA, or amplified genomic DNA.

For instance, polymorphisms in DNA sequences can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods, for example as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523, 2003; Cui et al., Bioinformatics 21:3852-3858, 2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening of a plurality of polymorphisms. Typing of target sequences by microarray-based methods is described in U.S. Pat. Nos. 6,799,122; 6,913,879; and 6,996,476.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR, forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′ à 3′ exonuclease activity cleaves the probe and separates the reporter dye from the quencher dye resulting in increased fluorescence of the reporter.

In another embodiment, a locus or loci of interest can be directly sequenced using nucleic acid sequencing technologies. Methods for nucleic acid sequencing are known in the art and include technologies provided by 454 Life Sciences (Branford, CT), Agencourt Bioscience (Beverly, MA), Applied Biosystems (Foster City, CA), LI-COR Biosciences (Lincoln, NE), NimbleGen Systems (Madison, WI), Illumina (San Diego, CA), and VisiGen Biotechnologies (Houston, TX). Such nucleic acid sequencing technologies comprise formats such as parallel bead arrays, sequencing by ligation, capillary electrophoresis, electronic microchips, “biochips,” microarrays, parallel microchips, and single-molecule arrays.

Various genetic engineering technologies have been developed and may be used by those of skill in the art to introduce traits in plants. In certain aspects, traits are introduced into cucumber plants via altering or introducing a single genetic locus or transgene into the genome of a variety or progenitor thereof. Methods of genetic engineering to modify, delete, or insert genes and polynucleotides into the genomic DNA of plants are well-known in the art.

In some embodiments, plant tissue of a plant comprising the CABYV resistance allele are also provided. The tissue can be undifferentiated tissue or already differentiated tissue. Undifferentiated tissue includes, for example, stem tips, anthers, petals, and pollen. This tissue can be used in micropropagation to obtain new plantlets that are grown into new plants comprising the CABYV resistance allele. The tissue can also be grown from a cell of the cucumber plants described herein. In certain embodiments, a tissue culture of a cucumber plant resistant to CABYV is also provided. Such tissue culture can be selected or derived from any part of the plant, in particular, from leaves, pollen, embryos, cotyledon, hypocotyls, meristematic cells, roots, root tips, anthers, flowers, seeds, and stems. The tissue culture can be regenerated into a cucumber plant comprising the CABYV resistance allele described herein, wherein the regenerated cucumber plant expresses the CABYV resistance trait.

In specific embodiments, improved cucumber lines can be created through the site-specific modification of a plant genome. Methods of genetic engineering include, for example, utilizing sequence-specific nucleases such as zinc-finger nucleases (see, for example, U.S. Pat. Appl. Pub. No. 2011-0203012); engineered or native meganucleases; TALE-endonucleases (see, for example, U.S. Pat. Nos. 8,586,363 and 9,181,535); and RNA-guided endonucleases, such as those of the CRISPR/Cas systems (see, for example, U.S. Pat. Nos. 8,697,359 and 8,771,945 and U.S. Pat. Appl. Pub. No. 2014-0068797). One embodiment of the disclosure thus relates to utilizing a nuclease or any associated protein to carry out genome modification. This nuclease could be provided heterologously within donor template DNA for templated-genomic editing or in a separate molecule or vector. A recombinant DNA construct may also comprise a sequence encoding one or more guide RNAs to direct the nuclease to the site within the plant genome to be modified. Further methods for altering or introducing a single genetic locus include, for example, utilizing single-stranded oligonucleotides to introduce base pair modifications in a cucumber plant genome (see, for example Sauer et al., Plant Physiol, 170(4):1917-1928, 2016).

Methods for site-directed alteration or introduction of a single genetic locus are well-known in the art and include those that utilize sequence-specific nucleases, such as the aforementioned, or complexes of proteins and guide-RNA that cut genomic DNA to produce a double-strand break (DSB) or nick at a genetic locus. As is well-understood in the art, during the process of repairing the DSB or nick introduced by the nuclease enzyme, a donor template, transgene, or expression cassette polynucleotide may become integrated into the genome at the site of the DSB or nick. The presence of homology arms in the DNA to be integrated may promote the adoption and targeting of the insertion sequence into the plant genome during the repair process through homologous recombination or non-homologous end joining (NHEJ).

In another embodiment of the present disclosure, genetic transformation may be used to insert a selected transgene into a plant or may, alternatively, be used for the preparation of transgenes which can be introduced by backcrossing. Methods for the transformation of plants that are well-known to those of skill in the art and applicable to many crop species include, but are not limited to, electroporation, microprojectile bombardment, Agrobacterium-mediated transformation, and direct DNA uptake by protoplasts.

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wound tissues in a controlled manner.

An efficient method for delivering transforming DNA segments to plant cells is microprojectile bombardment. In this method, particles are coated with nucleic acids and delivered into cells by a propelling force. Exemplary particles include those comprised of tungsten, platinum, and preferably, gold. For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target cells. The screen disperses the particles so that they are not delivered to the recipient cells in large aggregates. Microprojectile bombardment techniques are widely applicable and may be used to transform virtually any plant species.

Agrobacterium-mediated transfer is another widely applicable system for introducing gene loci into plant cells. An advantage of the technique is that DNA can be introduced into whole plant tissues, thereby bypassing the need for regeneration of an intact plant from a protoplast. Modern Agrobacterium transformation vectors are capable of replication in E. coli as well as Agrobacterium, allowing for convenient manipulations (Klee et al., Nat. Biotechnol., 3(7):637-642, 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti genes can be used for transformation.

In those plant strains where Agrobacterium-mediated transformation is efficient, it is the method of choice because of the facile and defined nature of the gene locus transfer. The use of Agrobacterium-mediated plant integrating vectors to introduce DNA into plant cells is well known in the art (Fraley et al., Nat. Biotechnol., 3:629-635, 1985; U.S. Pat. No. 5,563,055).

Transformation of plant protoplasts also can be achieved using methods based on calcium phosphate precipitation, polyethylene glycol treatment, electroporation, and combinations of these treatments (see, for example, Potrykus et al., Mol. Gen. Genet., 199:183-188, 1985; Omirulleh et al., Plant Mol. Biol., 21(3): 415-428, 1993; Fromm et al., Nature, 312:791-793, 1986; Uchimiya et al., Mol. Gen. Genet., 204:204, 1986; Marcotte et al., Nature, 335:454, 1988). Transformation of plants and expression of foreign genetic elements is exemplified in Choi et al. (Plant Cell Rep., 13:344-348, 1994), and Ellul et al. (Theor. Appl. Genet., 107:462-469, 2003).

### V. DEFINITIONS

The following definitions are provided to better define the present invention and to guide those of ordinary skill in the art in the practice of the present invention. Unless otherwise noted, terms are to be understood according to conventional usage by those of ordinary skill in the relevant art.

As used herein, the term “plant” includes plant cells, plant protoplasts, plant cells of tissue culture from which cucumber plants can be regenerated, plant calli, plant clumps and plant cells that are intact in plants or parts of plants such as pollen, flowers, seeds, leaves, stems, and the like.

As used herein, the term “population” means a genetically heterogeneous collection of plants that share a common parental derivation.

As used herein, the terms “variety” and “cultivar” mean a group of similar plants that by their genetic pedigrees and performance can be identified from other varieties within the same species.

As used herein, an “allele” refers to one of two or more alternative forms of a genomic sequence at a given locus on a chromosome.

A “quantitative trait locus” (QTL) is a chromosomal location that encodes for at least a first allele that affects the expressivity of a phenotype.

As used herein, a “marker” means a detectable characteristic that can be used to discriminate between organisms. Examples of such characteristics include, but are not limited to, genetic markers, biochemical markers, metabolites, morphological characteristics, and agronomic characteristics.

As used herein, the term “phenotype” means the detectable characteristics of a cell or organism that can be influenced by gene expression.

As used herein, the term “genotype” means the specific allelic makeup of a plant.

As used herein, “elite” or “cultivated” variety means any variety that has resulted from breeding and selection for superior agronomic performance. An “elite plant” refers to a plant belonging to an elite variety. Numerous elite varieties are available and known to those of skill in the art of cucumber breeding. An “elite population” is an assortment of elite individuals or varieties that can be used to represent the state of the art in terms of agronomically superior genotypes of a given crop species, such as cucumber. Similarly, an “elite germplasm” or elite strain of germplasm is an agronomically superior germplasm.

As used herein, the term “introgressed,” when used in reference to a genetic locus, refers to a genetic locus that has been introduced into a new genetic background, such as through backcrossing. Introgression of a genetic locus can be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

As used herein, the terms “recombinant” or “recombined” in the context of a chromosomal segment refer to recombinant DNA sequences comprising one or more genetic loci in a configuration in which they are not found in nature, for example as a result of a recombination event between homologous chromosomes during meiosis.

As used herein, the term “linked,” when used in the context of nucleic acid markers and/or genomic regions, means that the markers and/or genomic regions are located on the same linkage group or chromosome such that they tend to segregate together at meiosis.

As used herein, “tolerance locus” means a locus associated with tolerance or resistance to disease. For instance, a tolerance locus according to the present disclosure may, in one embodiment, control tolerance or susceptibility to CABYV.

As used herein, “tolerance” or “improved tolerance” in a plant refers to the ability of the plant to perform well, for example by maintaining yield, under disease conditions. Tolerance may also refer to the ability of a plant to maintain a plant vigor phenotype under disease conditions. Tolerance is a relative term, indicating that a “tolerant” plant is more able to maintain performance compared to a different (less tolerant) plant (e.g. a different plant variety) grown in similar disease conditions. One of skill will appreciate that plant tolerance to disease conditions varies widely, and can represent a spectrum of more-tolerant or less-tolerant phenotypes. However, by simple observation, one of skill can generally determine the relative tolerance of different plants, plant varieties, or plant families under disease conditions, and furthermore, will also recognize the phenotypic gradations of “tolerance.”

As used herein “resistance” or “improved resistance” in a plant to disease conditions is an indication that the plant is more able to reduce disease burden than a non-resistant or less resistant plant. Resistance is a relative term, indicating that a “resistant” plant is more able to reduce disease burden compared to a different (less resistant) plant (e.g., a different plant variety) grown in similar disease conditions. One of skill will appreciate that plant resistance to disease conditions varies widely, and can represent a spectrum of more-resistant or less-resistant phenotypes. However, by simple observation, one of skill can generally determine the relative resistance of different plants, plant varieties, or plant families under disease conditions, and furthermore, will also recognize the phenotypic gradations of “resistant.”

As used herein, “resistance allele” means the nucleic acid sequence associated with tolerance or resistance to disease. The use of the term “a resistance allele” does not exclude a genomic region that comprises more than one gene or other genetic factor. Specifically, a “disease resistance allele” can denote a haplotype allele within a haplotype window or genomic region wherein a phenotype associated with the haplotype allele can be disease resistance. A haplotype window is a contiguous genomic region that can be defined, and tracked, with a set of one or more polymorphic markers wherein the polymorphisms indicate identity by descent. A haplotype within that window can be defined by the unique fingerprint of alleles at each marker. When all the alleles present at a given locus on a chromosome are the same, that plant is homozygous at that locus. If the alleles present at a given locus on a chromosome differ, that plant is heterozygous at that locus. Plants may be homozygous or heterozygous at any particular resistance locus or for a particular polymorphic marker.

As used herein, “polymorphism” means the presence of one or more variations of a nucleic acid sequence at one or more loci in a population of one or more individuals. The variation may comprise but is not limited to one or more base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and a tag SNP. A genetic marker, a gene, a DNA-derived sequence, a haplotype, an RNA-derived sequence, a promoter, a 5′ untranslated region of a gene, a 3′ untranslated region of a gene, microRNA, siRNA, a QTL, a satellite marker, a transgene, mRNA, ds mRNA, a transcriptional profile, and a methylation pattern may also comprise polymorphisms.

As used herein, the term “haplotype” means a chromosomal region within a haplotype window defined by at least one polymorphic molecular marker. The unique marker fingerprint combinations in each haplotype window define individual haplotypes for that window. Further, changes in a haplotype, brought about by recombination for example, may result in the modification of a haplotype so that it comprises only a portion of the original (parental) haplotype operably linked to the trait, for example, via physical linkage to a gene, QTL, or transgene. Any such change in a haplotype would be included in the definition of what constitutes a haplotype so long as the functional integrity of that genomic region is unchanged or improved.

As used herein, the term “haplotype window” means a chromosomal region that is established by statistical analyses known to those of skill in the art and is in linkage disequilibrium. Thus, identity by state between two inbred individuals (or two gametes) at one or more molecular marker loci located within this region is taken as evidence of identity-by-descent of the entire region. Each haplotype window includes at least one polymorphic molecular marker. Haplotype windows can be mapped along each chromosome in the genome. Haplotype windows are not fixed per se and, given the ever-increasing density of molecular markers, this disclosure anticipates the number and size of haplotype windows to evolve, with the number of windows increasing and their respective sizes decreasing, thus resulting in an ever-increasing degree confidence in ascertaining identity by descent based on the identity by state at the marker loci.

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits.

### VI. DEPOSIT INFORMATION

A deposit was made of at least 625 seeds of a cucumber line that comprises the CABYV resistance allele on chromosome 4, as described herein. The deposit was made with the with Provasoli-Guillard National Center for Marine Algae and Microbiota (NCMA), 60 Bigelow Drive, East Boothbay, Maine, 04544 USA. The deposit is assigned NCMA Accession Number 202409001, and the date of deposit was Sep. 6, 2024. Upon issuance of a patent, all restrictions upon the deposit will be removed, and the deposit is intended to meet all of the requirements of 37 C.F.R. §§ 1.801-1.809. The deposit has been accepted under the Budapest Treaty and will be maintained in the depository for a period of 30 years, 5 years after the last request, or the effective life of the patent, whichever is longer, and will be replaced if necessary during that period. Applicants do not waive any infringement of their rights granted under this patent or any other form of variety protection, including the Plant Variety Protection Act (7 U.S.C. 2321 et seq.)

### EXAMPLES

### Example 1. A CABYV Resistance Locus was Identified in Cucumber on Chromosome 4

Under outdoor open field conditions, a diversity panel composed of cucumber elite lines and landraces was naturally inoculated by wild aphid populations. The incidence of CABYV was determined with ELISA to determine the incidence of CABYV on this diversity panel population of cucumbers. The presence of CABYV was tested by ELISA in each plant sixfold throughout the crop cycle as long as the plants were alive and CABYV-negative.

A genome-wide association study (GWAS) was used on the phenotypic and genotypic data collected from the diversity panel. Two methods were used to detect QTLs: multi-locus mixed-model (MLMM) and local score approach (LSA), with the latter being utilized specifically to identify low effect QTLs. Accordingly, LSA enabled the mapping of low effect QTLs and confirmed QTLs that barely reached the significance threshold through conventional MLMM GWAS. MLMM is an iterative GWAS approach that selects the most significant SNPs from the previous steps and adds them as a fixed effect in the GWAS model of the following step, thus enabling the detection of additive SNPs. The MLMM model described in Segura et al. (Nat Genet. 44:825-830, 2012) was used to perform GWAS. Five models were tested using PoPs as aggregated phenotypes: kinship (K), genetic structure (Q5), genetic structure (Q9), kinship+genetic structure (KQ5), and kinship+genetic structure (KQ9). The predicted genetic values per accession were obtained using a Poisson generalized linear model and called Poisson BLUP (POP). MLMM was applied on PoPs using the model:

$$y = {X}_{M} β + {X}_{w} v + g + e$$

where y is the phenotype vector (PoPs); β is the vector of allelic effects; X_{M} is the genotypic matrix of all accessions at the SNP being tested; v is the fixed effect vector; including the population genetic structure and top SNP, depending on the model and MLMM step; X_{W} is the fixed effect incidence matrix; g is the random genotype effect vector and

$$g \to N ( 0 , K {σ}_{g}^{2} ) ;$$

and e is the error vector

$$e \to N ( 0 , K {σ}_{e}^{2} ) .$$

Both g and e are assumed to be independent.

The significance threshold was set for the CABYV maximum infection rate with a Bonferroni threshold corrected by the number of independent SNPs identified with the simpleM method. P-values obtained in the MLMM first step via the model that included both the kinship and nine genetic groups (KQ9) generated the curve that best fit the bisector on the QQplot. This model reflected a quasi-uniform distribution of the p-values for most SNPs, as expected under the hypothesis that only a few markers are likely associated to QTLs. QTLs were delineated by a local linkage disequilibrium (LD) approach and borders were set when no further SNPs with r²>0.2 with respect to the top SNP was found.

The local score approach (LSA) was initially developed to detect selection signatures by scanning forward and backward SNPs in the genome using Fst parameters. It was successfully translated to detect low effect QTLs by scanning SNPs in the genome using p-values collected by GWAS. For the calculation of the local score, a conservative value of ξ=2 was chosen and the genomic inflation factor λ_{GC} was calculated. When λ_{GC}>1.001, p-values were resampled to set the significance thresholds.

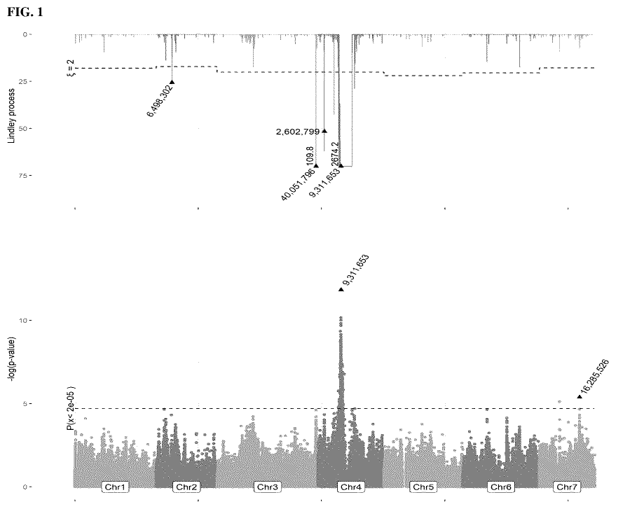

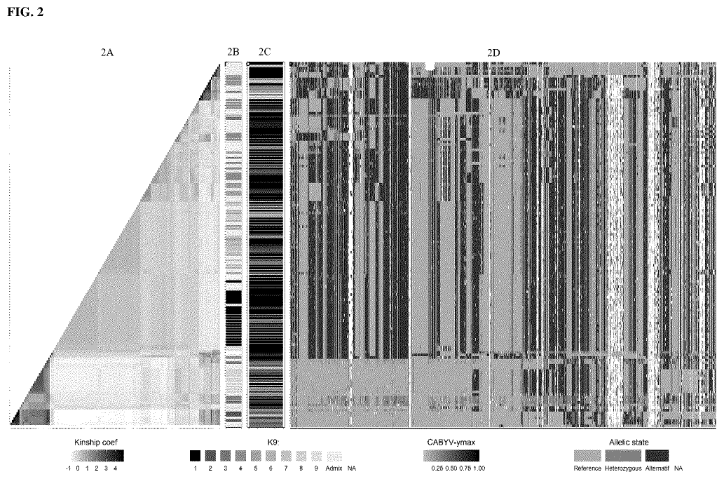

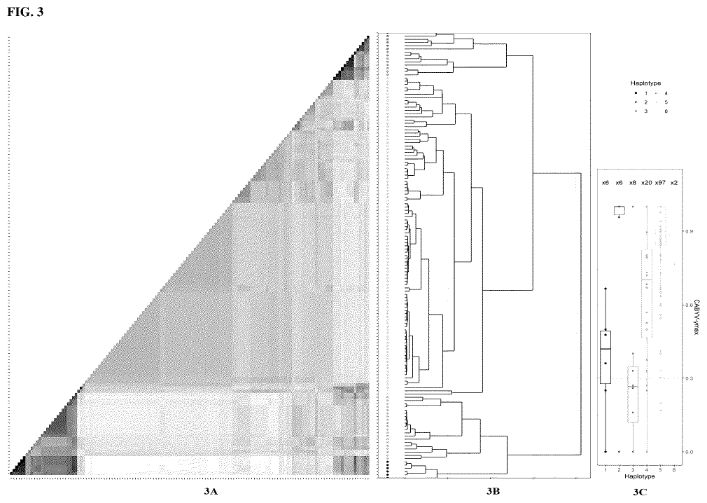

Five QTLs were detected for the CABYV maximum infection rate trait and their intervals and related top SNP information, including physical position, minor allele frequencies (MAF), resistance allele frequencies within each genetic group, are summarized in Tables 1 and 2 below and in FIGS. 1-3.

TABLE 1
QTLs identified for CABYV Max Infection Rate trait - KQ9 Model

| GWAS | | | | SNP | Begin LD | End LD | Width LD |
|---|---|---|---|---|---|---|---|
| Method | QTL | Chr | Type | number | (bp) | (bp) | (bp) |
| LocalScore | QTL3 | Chr4 | Peak | 111 | 2,524,830 | 2,829,252 | 304,422 |
| MLMM | QTL5 | Chr7 | Single | 1 | 15,185,443 | 16,358,161 | 1,172,718 |
| | | | SNP | | | | |
| Both | QTL4 | Chr4 | Peak | 748 | 8,682,572 | 10,599,858 | 1,917,286 |
| LocalScore | QTL2 | Chr3 | Peak | 201 | 37,993,813 | 40,877,092 | 2,883,279 |
| LocalScore | QTL1 | Chr2 | Peak | 135 | 6,415,831 | 6,703,566 | 287,735 |

TABLE 2
Top SNP identified for CABYV Max Infection Rate QTLs

| | | Lindley | | Top SNP | |
|---|---|---|---|---|---|
| Top SNP | Pval_step1 | Score | ζ | Position (bp) | MAF |
| Chr4_02602799 | 4.3 | 62.3 | 2 | 2,602,799 | 5.0% |
| Chr7_16285526 | 5.4 | | | 16,285,526 | 19.8% |
| Chr4_09311653 | 11.8 | 2222.8 | 2 | 9,311,653 | 24.1% |
| Chr3_40051796 | 4.6 | 109.8 | 2 | 40,051,796 | 25.9% |
| Chr2_06498302 | 2.6 | 27.3 | 2 | 6,498,302 | 5.0% |

A highly significant QTL that was about 2 Mb wide was detected on chromosome 4 by both methods. Its top SNP had a MAF=0.24 and the resistant allele was heterogeneously distributed among the nine genetic groups-two features that made this QTL very interesting. This QTL reduced the maximum infection rate of 42%. Two QTLs that were less significant, but which showed about the same average difference in maximum infection rate, were mapped on chromosome 7 by MLMM and on chromosome 3 by LSA. Two low effect QTLs were mapped by LSA on chromosomes 2 and 4. The CABYV resistance QTL identified has the expected peak shape, meaning that several mutations in the same genomic area are supporting the QTLs, contrary to a single mutation that could be potentially genotyping error or false positive. The minor allele frequency (MAF) denotes the frequency of the minor allele in a given population. Variants with MAF>5% are considered to be common. A MAF of 24.1% for chromosome 4, as shown in Table 2 above, is considered well-balanced, as it indicates that the favorable/unfavorable alleles are present in enough individuals to validate the QTL effect and it is not a rare allele.

The allelic state of each top SNP for each line was determined where 0=homozygous alternative, 1=heterozygous, 2=homozygous reference. For each allelic state of each top SNP, the frequency within the population (Freq), the average of infection rate (Avg), and the standard deviation (Std) was determined. The lower the standard deviation value, the higher the confidence that the average is representative of the group. The population screened was composed of 149 lines, each line being represented by 10 plants in the field. The infection rate was determined as the percentage of infected plants for each line, as shown in Table 3.

TABLE 3
Metrics for the different alleles of the top SNPs identified for CABYV max infection rate QTLs.

| TopSNP | Avg_0 | Std_0 | Freq_0 | Avg_1 | Std_1 | Freq_1 | Avg_2 | Std_2 | Freq_2 |
|---|---|---|---|---|---|---|---|---|---|
| Chr4_02602799 | 39.0% | 36.8% | 4.3% | 81.8% | 25.8% | 1.4% | 80.4% | 27.2% | 94.2% |
| Chr7_16285526 | 48.3% | 29.6% | 15.8% | 72.7% | 22.7% | 7.9% | 85.6% | 24.8% | 76.3% |
| Chr4_09311653 | 87.9% | 19.8% | 73.4% | 81.4% | 22.5% | 5.0% | 46.5% | 32.8% | 21.6% |
| Chr3_40051796 | 83.0% | 24.3% | 22.3% | 67.7% | 37.4% | 7.2% | 78.4% | 29.0% | 70.5% |
| Chr2_06498302 | 90.1% | 17.2% | 5.0% | — | — | — | 78.1% | 29.1% | 95.0% |

Lines with the allelic state 0 at the QTL on chromosome 4 represent 73.4% of the general population and have an average infection rate of 87.9%, with a standard deviation of 19.8%. This indicates that 73.4% of the 149 lines have an infection rate around 90% (i.e. 9 infected plants over 10 in the end of the trial). Therefore, the allelic state 0 can be considered a susceptibility allele. In contrast, 21.6% of the population has allelic state 2 and has 46.5% of infection rate. This indicates that approximately half the plants of the line are free of CABYV while the other half infected is infected with CABYV and can be considered intermediate resistance. Therefore, the QTL on chromosome 4 flanked by marker locus M1 (SEQ ID NO:1) and marker locus M2 (SEQ ID NO:10) is identified as a QTL whose presence results in going from susceptibility (0-homozygous alternative) to intermediate resistance (2-homozygous reference).

### Example 2. A CABYV Resistance Locus was Identified in Cucumber on Chromosome 4 and Additional Methods for Selection Devised

Markers that associated with the CABYV resistance QTL on chromosome 4 of the public cucumber genome map version 3 Cucumber Chinese Long′ (CCL) landrace (Li et al., 2019) (Li et al. 2019) were identified and are provided in Table 4 below. These markers interrogate polymorphisms spread along the newly identified QTL and allow differentiation between resistant and susceptible, i.e. comprising the introgression or lacking the introgression, genotypes. The “favorable allele,” as denoted in Table 4, is associated with the presence of the CABYV resistance locus, introgression, and resistance phenotype.

TABLE 4
List of markers and favorable alleles at each marker for tracking resistance QTLs.

| | | | | SNP | | | Marker |
|---|---|---|---|---|---|---|---|
| | | Public | Marker | position in | | | Sequence |
| Marker | | Position | Size | marker | SNP | Favorable | (SEQ ID |
| Name | Chr. | (bp) | (bp) | (bp) | Change | Allele | NO) |
| M1 | 4 | 8,682,572 | 2000 | 1000 | [A/G] | A | 1 |
| M3 | 4 | 8,872,385 | 200 | 100 | [A/G] | A | 2 |
| M4 | 4 | 9,042,752 | 200 | 100 | [G/C] | G | 3 |
| M5 | 4 | 9,310,448 | 200 | 100 | [G/T] | G | 4 |
| M6 | 4 | 9,561,517 | 200 | 100 | [C/T] | C | 5 |
| M7 | 4 | 9,734,323 | 200 | 100 | [A/G] | A | 6 |
| M8 | 4 | 9,898,582 | 200 | 100 | [C/T] | C | 7 |
| M9 | 4 | 10,100,790 | 200 | 100 | [T/C] | T | 8 |
| M10 | 4 | 10,363,190 | 200 | 100 | [A/T] | A | 9 |
| M2 | 4 | 10,505,104 | 2000 | 1000 | [A/G] | A | 10 |

### Example 3. Validation of the CABYV Resistance QTL on Chromosome 4

A larger diversity panel comprising the diversity panel described in Example 1 above along with additional cucumber lines was tested in a bioassay developed for CABYV in cucumber to further define and validate the resistance QTL on chromosome 4 identified as highly significant. A total of 105 unique pedigrees comprising both elite and landraces were screened wherein two replicates of four plants were tested for each entry.

In the bioassay, the inoculum was produced by infecting susceptible cucumber plants with viruliferous aphids (Aphis gossypii). The aphids were allowed to feed on the plants for a period of 3-4 days to ensure adequate virus transmission. Following this, leaf pieces containing 10-30 viruliferous aphids were cut and placed on the first true leaf of the seedlings (11 days post sowing) to initiate the inoculation process. Aphids were removed from plants one week after placing the leaf pieces. Plants were planted in greenhouse 10 days post inoculation. The environmental conditions during the bioassay were controlled, with optimal temperatures maintained at 26° C. during the day and 20° C. at night.

Evaluation of the plants for CABYV infection was conducted weekly for up to 6 weeks post inoculation. The rating scale for evaluating the severity of symptoms included the following classes:
1: Healthy—No visible symptoms of infection.
2: Necrotic reaction—Yellow/orange spots turning necrotic, or necrotic veins present.
3: Mild yellowing—Less than 50% leaf surface affected, restricted to the first two leaves.
5: Intense yellowing—More than 50% leaf surface affected on the first leaves, with mild yellowing extending to upper leaves.
7: Intense yellowing—More than 50% leaf surface affected in the lower to mid part of the plant.
9: Severe yellowing—Severe yellowing in the lower to mid part of the plant, extending to the top.

A GWAS was performed on the phenotypic and genotypic data collected from the diversity panel. For each accession, the aggregated phenotypic value was calculated as a simple mean of the scores from the 8 plants trialed.

The single-locus method (Yu et al., 2006) was used to perform GWAS. The KQ2 model, which accounts for relatedness between accessions (based on the kinship (K) matrix) and for the population structure (based on a structure (Q) matrix with two genetic groups), was applied on the aggregated phenotype using the model:

$$y = {X}_{M} β + {X}_{W} v + g + e$$

where y is the phenotype vector; β is the vector of allelic effects; X_{M} is the genotypic matrix of all accessions at the SNP being tested; v is the fixed effect vector, including the population genetic structure and top SNP; X_{W} is the fixed effect incidence matrix; g is the random genotype effect vector and

$$g \to N ( 0 , K {σ}_{g}^{2} ) ;$$

and e is the error vector

$$e \to N ( 0 , K {σ}_{e}^{2} ) .$$

Both g and e are assumed to be independent. The significance threshold was set to 3.1 and is a Bonferroni threshold corrected by the number of independent SNPs identified with the simpleM method (Gao et al., 2008). The QTL was delineated by a local linkage disequilibrium (LD) approach and the interval boundaries were set when no further SNPs with r²>0.2 with respect to the top SNP were found.

The GWAS analysis on visual symptoms evaluated at 6 weeks post-inoculation resulted in the re-identification and validation of the QTL on chromosome 4. Its interval and associated SNP information, including physical position on chromosome 4 of the public cucumber genome map version 3 Cucumber Chinese Long′ (CCL) landrace (Li et al., 2019), are summarized in Table 5 below. The “favorable allele,” as denoted in Table 5, is associated with the presence of the CABYV resistance locus, introgression, and resistance phenotype.

TABLE 5
Additional markers associated with CABYV resistance QTL on chromosome 4.

| | | | | SNP | | | Marker |
|---|---|---|---|---|---|---|---|
| | | Public | Marker | position in | | | Sequence |
| Marker | | Position | Size | marker | SNP | Favorable | (SEQ ID |
| Name | Chr. | (bp) | (bp) | (bp) | Change | Allele | NO) |
| M11 | 4 | 9,205,907 | 122 | 61 | T/C | T | 11 |
| M12 | 4 | 9,744,356 | 107 | 61 | C/T | C | 12 |
| M13 | 4 | 9,876,485 | 1749 | 773 | A/G | G | 13 |

The reduced interval on chromosome 4 is defined by the left flanking marker M11, positioned at 9,205,907 bp and the right flanking marker M13, positioned at 9,876,485 bp, and spans a total of 669,866 base pairs. The peak marker identified in the reduced interval, marker M12, is located at 9,744,356 bp on chromosome 4. This marker has a p-value of 4.4, indicating high significance, and therefore passes the significance threshold of 3.1. The minor allele frequencies (MAF) and resistance allele frequencies for marker M12 are summarized Table 6 below.

TABLE 6
Allele frequency and efficacy for CABYV resistance QTL on chromosome 4.

| Alt (0) | Ref (2) | MAF | Freq_allele_0 | Freq_allele_2 | AdjR 2 | Mean_0 | Std_0 | Mean_2 | Std_2 |
|---|---|---|---|---|---|---|---|---|---|
| T | C | 0.16 | 0.83 | 0.16 | 0.30 | 4.55 | 1.71 | 1.56 | 0.87 |

Sixteen percent of the panel described carry the resistance allele at this locus. Lines carrying the QTL on chromosome 4, which include diverse cucumber germplasm, exhibit a strong reduction of 2.99 in disease score on a rating scale of 1 to 9. Lines that showed very low to no infection with CABYV in the open field assay did not develop CABYV symptoms in the bioassay or exhibited only very mild symptoms.

The interval identified in the validation experiment of Example 3 co-locates and is positioned within the region on chromosome 4 described in Example 1. The strong correlation between resistance behavior observed in the open field assay and the bioassay, combined with the overlapping QTL intervals identified on chromosome 4, validates the identification of a QTL that confers resistance to CABYV with a significant effect, across different cucumber germplasm.

What is claimed is:

1. A method of producing a cucumber plant exhibiting resistance to cucurbit aphid-borne yellows virus (CABYV), comprising introgressing into a plant a CABYV resistance allele within a chromosomal segment flanked in the genome of said plant by marker locus M1 (SEQ ID NO:1) and marker locus M2 (SEQ ID NO:10) on chromosome 4, wherein said introgressed CABYV resistance allele confers to said plant increased resistance to CABYV compared to a plant not comprising said allele.

2. The method of claim 1, wherein said introgressing comprises:
a) crossing a cucumber plant comprising said chromosomal segment with itself or with a second cucumber plant to produce one or more progeny plants; and
b) selecting a progeny plant comprising said chromosomal segment.

3. The method of claim 2, wherein a representative sample of seed comprising said chromosomal segment has been deposited under NCMA Accession Number 202409001.

4. The method of claim 1, wherein said chromosomal segment comprises a marker locus on chromosome 4 selected from the group consisting of marker locus M1 (SEQ ID NO:1), marker locus M3 (SEQ ID NO:2), marker locus M4 (SEQ ID NO:3), marker locus M5 (SEQ ID NO:4), marker locus M6 (SEQ ID NO:5), marker locus M7 (SEQ ID NO:6), marker locus M8 (SEQ ID NO: 7), marker locus M9 (SEQ ID NO:8), marker locus M10 (SEQ ID NO:9), marker locus M11 (SEQ ID NO:11), marker locus M12 (SEQ ID NO:12), marker locus M13 (SEQ ID NO:13), and marker locus M2 (SEQ ID NO:10).

5. The method of claim 1, wherein said introgressing comprises backcrossing, marker-assisted selection, or assaying for said CABYV resistance.

6. A cucumber plant produced by the method of claim 1, wherein said plant comprises said recombinant chromosomal segment.

7. A method for selecting a cucumber plant with increased resistance to cucurbit aphid-borne yellows virus (CABYV), comprising:
(a) crossing a cucumber plant comprising a CABYV resistance allele with a second cucumber plant to produce a population of progeny plants; and
(b) selecting a progeny plant comprising said CABYV resistance allele;
wherein selecting said progeny plant comprises detecting a marker locus within or genetically linked to a chromosomal segment flanked in the genome of said plant by marker locus M1 (SEQ ID NO:1) and marker locus M2 (SEQ ID NO:10) on chromosome 4.

8. The method of claim 7, wherein selecting a progeny plant comprises detecting nucleic acids comprising marker locus M1 (SEQ ID NO:1), marker locus M3 (SEQ ID NO:2), marker locus M4 (SEQ ID NO:3), marker locus M5 (SEQ ID NO:4), marker locus M6 (SEQ ID NO:5), marker locus M7 (SEQ ID NO:6), marker locus M8 (SEQ ID NO:7), marker locus M9 (SEQ ID NO:8), marker locus M10 (SEQ ID NO:9), marker locus M11 (SEQ ID NO:11), marker locus M12 (SEQ ID NO: 12), marker locus M13 (SEQ ID NO:13), and marker locus M2 (SEQ ID NO:10).

9. The method of claim 7, wherein the progeny plant is an F₂-F₆ progeny plant.

10. The method of claim 7, wherein producing said progeny plant comprises backcrossing.

11. A method of selecting a cucumber plant exhibiting resistance to cucurbit aphid-borne yellows virus (CABYV), comprising:
a) obtaining a population of progeny plants having a parent comprising resistance to CABYV;
b) screening said population with at least one nucleic acid marker to detect a polymorphism genetically linked to CABYV resistance; and
c) selecting from said population one or more progeny plants comprising a haplotype associated with CABYV resistance, wherein the haplotype comprises a CABYV resistance allele flanked in the genome of said plant by marker locus M1 (SEQ ID NO:1) and marker locus M2 (SEQ ID NO: 10) on chromosome 4.

12. The method of claim 11, wherein selecting said one or more progeny plants comprises:
(a) detecting a marker locus within or genetically linked to said chromosomal segment flanked in the genome of said plant by marker locus M1 (SEQ ID NO:1) and marker locus M2 (SEQ ID NO:10) on chromosome 4; or
(b) detecting at least one polymorphism at a locus selected from the group consisting of marker locus M1 (SEQ ID NO:1), marker locus M3 (SEQ ID NO:2), marker locus M4 (SEQ ID NO:3), marker locus M5 (SEQ ID NO:4), marker locus M6 (SEQ ID NO:5), marker locus M7 (SEQ ID NO:6), marker locus M8 (SEQ ID NO:7), marker locus M9 (SEQ ID NO:8), marker locus M10 (SEQ ID NO:9), marker locus M11 (SEQ ID NO:11), marker locus M12 (SEQ ID NO:12), marker locus M13 (SEQ ID NO:13), and marker locus M2 (SEQ ID NO:10).

13. The method of claim 11, wherein said progeny plant is an F₂-F₆ progeny plant.

14. The method of claim 11, wherein producing said progeny plant comprises backcrossing.

15. The method of claim 11, wherein screening said population comprises PCR, single strand conformational polymorphism analysis, denaturing gradient gel electrophoresis, cleavage fragment length polymorphism analysis, TAQMAN assay, and/or DNA sequencing.

16. The method of claim 11, wherein a representative sample of seed comprising said allele has been deposited under NCMA Accession Number 202409001.

17. A method of selecting a cucumber plant exhibiting resistance to cucurbit aphid-borne yellows virus (CABYV), comprising:
a) screening one or more plants with at least one nucleic acid marker to detect a polymorphism genetically linked to CABYV resistance; and
b) selecting one or more plants comprising a haplotype associated with CABYV resistance, wherein the haplotype comprises a CABYV resistance allele flanked in the genome of said plant by marker locus M1 (SEQ ID NO:1) and marker locus M2 (SEQ ID NO:10) on chromosome 4.

18. The method of claim 17, wherein the CABYV resistance allele is further defined as located within a chromosomal segment flanked in the genome of said plant by marker locus M1 (SEQ ID NO: 1) and marker locus M2 (SEQ ID NO:10) on chromosome 4.

19. The method of claim 17, wherein selecting one or more plants comprises:
(a) detecting a marker locus within or genetically linked to a chromosomal segment flanked in the genome of said plant by marker locus M1 (SEQ ID NO:1) and marker locus M2 (SEQ ID NO:10) on chromosome 4; or
(b) detecting at least one polymorphism at a locus selected from the group consisting of marker locus M1 (SEQ ID NO:1), marker locus M3 (SEQ ID NO:2), marker locus M4 (SEQ ID NO:3), marker locus M5 (SEQ ID NO:4), marker locus M6 (SEQ ID NO:5), marker locus M7 (SEQ ID NO:6), marker locus M8 (SEQ ID NO:7), marker locus M9 (SEQ ID NO:8), marker locus M10 (SEQ ID NO:9), marker locus M11 (SEQ ID NO:11), marker locus M12 (SEQ ID NO:12), marker locus M13 (SEQ ID NO:13), and marker locus M2 (SEQ ID NO:10).

20. The method of claim 17, wherein screening one or more plants comprises PCR, single strand conformational polymorphism analysis, denaturing gradient gel electrophoresis, cleavage fragment length polymorphism analysis, TAQMAN assay, and/or DNA sequencing.

21. A method for identifying a cucumber plant comprising a cucurbit aphid-borne yellows virus (CABYV) resistance allele comprising:
a) obtaining nucleic acids from at least a first cucumber plant; and
b) identifying in said nucleic acids the presence of at least a first genetic marker indicative of the presence of a chromosomal segment flanked in the genome of said plant by marker locus M1 (SEQ ID NO:1) and marker locus M2 (SEQ ID NO:10) on chromosome 4, wherein said CABYV resistance allele confers to said plant increased resistance to CABYV compared to a plant not comprising said allele.

22. The method of claim 21, wherein said identifying comprises detecting a marker genetically linked to marker locus M1 (SEQ ID NO:1), marker locus M3 (SEQ ID NO:2), marker locus M4 (SEQ ID NO:3), marker locus M5 (SEQ ID NO:4), marker locus M6 (SEQ ID NO:5), marker locus M7 (SEQ ID NO:6), marker locus M8 (SEQ ID NO:7), marker locus M9 (SEQ ID NO:8), marker locus M10 (SEQ ID NO:9), marker locus M11 (SEQ ID NO:11), marker locus M12 (SEQ ID NO: 12), marker locus M13 (SEQ ID NO:13), and marker locus M2 (SEQ ID NO:10).


SEQUENCE LISTING (13 sequences)

SEQ ID NO: 1 - DNA, 2000 bp, Cucumis sativus
  source 1..2000: mol_type = genomic DNA
gaacttaggataaatttataacaaaaaaccaaaaatttagaccttaacaatgaaatgcaattcagatgactgtaagacctactatcattcacttccatcctgttaatacacaacagggtacctttggtggtagtgagtaaacagtaaatttgggattagaaagttccaaaaatataaatcaaattgcatttctattaagcattcataactttaaaaaaaaaaaaaacatgtgtttcatggattcctcccacaatttggctgtggaacatggagaaacaatatattttcaactaacacagaaaagaaataaaacgaaaagtagcaaagttacccagcaaacaaccggaaagaaagtgaaattttcctttgcccatattttacacaagtaaaaagattcttcattttgaatatgtttttccaaattgaactgcagctggattatactaatatcataaagtacacataattaatcaagttactatgtgccactgttcacaaaaaaacattcttatatattgacaaaaagttgcaaaaaccatgattgcttcaagagattgacaataatcactaaaacaagaaataagaacaatattcttgtggaaaaaaacacccacataaacaagcatcccagtaagctaaaatgaatgtcaaaagcaactaacagaatgcagctgtaacaattgctatgccatctatatttgaattcattgaccactaccatacctaataagctacatcactttcccccgccgaattgcaataaattcaatatctccatcattaggaatgcaaaaaatcccaaattggccaaacaaagcaagatcatgggtacataattaaggggggaaaatctttattggtatgtggccttttgggttaaatcaaaaggtaaggtatgagggcttatgcctgaagtggacaaatatcataccattttcagagacgtatgtcctaactacaaagtcatgatctaaacctagtcatatttgtagaatccacrtgtgttggaaaangatctaatgaggctcaataacagtcatgcatagatctatggttgaactctattagataggggatgtgctccgaaaagaaaagcgatgacctcgtcaaaagctggagaggtcgatagctatttgagtcttgttggaaatgcaaagtctcacattggcaacacatgaggactattacaaatacttaaataatagaaaatatctccttggatgaggacattttggtgaaacaaaagacggattcatcaggtcttctaccaaattagcaatatcatttcatagagaaactgtgaagaggtcccttgtcctaacaattggcattagagggtatttgataatatagggttccatttcaaaaccacttgacaattagaatagtggctcatctatcttattaggagcgtgaggtcatttgatctttccaatgtgagattctcaacatgtctcctcaagatggtgttctcttttgggttcaccatttgtaatctctatcatattagataacatggagttccgtctcaaaaccaattggcaaatgagagtccatctatcttattaggagtatccggtcccttgattttccaatgcgggattctcaatagagacatgccccaagcttggtcatgtttgtaanatcccccagcatcaaggagagcaatatgtctcaggaacaaattgcatgctgtctaagaagaacgataagttccacagaatcctcaaaggtggtgagcttcaatagcatagtcgtgtggagatccgttgttgacagaggaaggggaaaaacctttaagttacctcaagagcagagtgtgtaagttttcattttccaatattaaagacattacacaaccaacgacacaaaatattacattccataaaaagtaaatgatggatgagcaccatgcgatcaattcccaaccntattgtaccatttcaaacaaaaataacgttaacacattacagagtaagtaaaggggaagaaagaggaca

SEQ ID NO: 2 - DNA, 200 bp, Cucumis sativus
  source 1..200: mol_type = genomic DNA
gggtgtgataattattgaagatcctagaaataagtatttaatgtcaaatcaataactcanaactaagtttgaggccaagactaaaaatgaattaaagaargtacgcatcaataccggtatccatatgagcatagaattttntnnnnnnttttttatgaataggtgtgtgctnctttactatatcaaacaaaaatttggtt

SEQ ID NO: 3 - DNA, 200 bp, Cucumis sativus
  source 1..200: mol_type = genomic DNA
ttaagtaagaatcatttttattcaactttttaatgaaagttatgtagaatgcacatggaaaatcaaaattattagaatttttcatttttttttcttcaasctaacaacactatttgaaaacatcttattattttgataaagttttaaaattgaaatgtgtgaaattaaaatttttaaagttagaaactaaattggattgt

SEQ ID NO: 4 - DNA, 200 bp, Cucumis sativus
  source 1..200: mol_type = genomic DNA
attacatgttgtatgtcttaaagataccaattcttagtggctatataattccaggttgcttattaaatagtgacattttgtgggttttgtaagtcacacktatgggtgaaacattacataaatgagggaaagtggattgttcacgtgagatttctaaagaaatttaattcgtggagtcaaactagtgttaatgttgtatt

SEQ ID NO: 5 - DNA, 200 bp, Cucumis sativus
  source 1..200: mol_type = genomic DNA
taaattaaatagtgatttaacaaatataatgtaaaagaatattccaagaataaccttggagtatcacttaaattttcaaaacgacctaataatgctaaaytataagctcataactcataacctatcaaaaaagtacagataaaacgaacaaactatttaaatcttgaaaaatgaaatatgagaaaaaaaaatgaaaagag

SEQ ID NO: 6 - DNA, 200 bp, Cucumis sativus
  source 1..200: mol_type = genomic DNA
ataattccttcaaacccgacaaatccctacaaaattaaacgaaatttttgttaggatgagatgtatgaacattgggagggaaaaaactaccccatcgacracatatttaaatagaagtttttgctcctcctactcaatattgcaatagcacaaaattaaaactcgtttgaatatattatatgacataattgatcaacact

SEQ ID NO: 7 - DNA, 200 bp, Cucumis sativus
  source 1..200: mol_type = genomic DNA
agaagaatataaacaaattatagtctcatttaatttgaagcacacntaaacactcatcttcatttttcatcttccatctttgatcttcaagtctacaacytgcaatctttagcatttgggattgaagaacaaaacccttcattcggaagaacaaaggtacaaaagttgcggcttctttctntcttaggggagattttggc

SEQ ID NO: 8 - DNA, 200 bp, Cucumis sativus
  source 1..200: mol_type = genomic DNA
ctttatatatccattacaaaagaatagagaatactaattaattacaacanaatcacaaacgtcaacttatctaacagaccaacngatcattttaaaatgycaagctctgtgatggataaaagaaattaactttcaaataactaaatttggccaacatgaacttaagctcaattggcaccatgttcctatggtcaagaggt

SEQ ID NO: 9 - DNA, 200 bp, Cucumis sativus
  source 1..200: mol_type = genomic DNA
aaatgttttccaaaaattcatttttatttaaatacttcctaaaataaagacacaaatgtgtttggcaatactattttaaaagtgtttttatttttttttwaaaaaatcaagtttgatcggtggcaacaatcgctcaaagtttgtcggtgaagtcacaaaaaataatagttagagattgacttcaatggtcaacaaaaatg

SEQ ID NO: 10 - DNA, 2000 bp, Cucumis sativus
  source 1..2000: mol_type = genomic DNA
tattatctactttttagttggaagtgcaaattaaagtacatttgttgtatagtttcactcaaaaaatgagctatttaccaatgatttttctagtgtatagttaatgtggggacttaaagcattccccaccaaattgagaagaataggattctgatttgaaggggaaaaaaataatctatttactattgtaaaggaggaaaaaagggcattggacacaaaaactcaatacatattttgtacaatatatatgatcatgcaataaaacatatccaaatttgatatggaatgtgaacagccaacttctatatatgcactactctattaatggtttttatattattcttttgtcttcaaagattcactttctgaaattggaaaaataaggattaaaaagtttgaataatataaagttttttattttgaattctttgtgtgtacatggagagattttgtacaataataaatgtttgaaaaatatagcacacaatgtgtttccttatgtaccttgcttttgttgtcttactacatctctttgcttcttctcttatcttagttatgtaaacaaagtatatatatatatatatagatcgattatttaatcgtctgtgtatattatatgatttaaggacgacaatgggccaacgtccactgggaaaaaaagagtttctatgacattgtatagctaaaccaaactaagaggagaaaaaaattaaatgtgctataaaattttgaagaaaaggtgctcttttttatttatttgtgtttttgaaccattacgacaatttttagcgtcgtcaagtgtataacgaacttttattatgttgagaattttttattgaaaaaatcaaggcgactcacgcttgatataaggtagatggactacttatctcattcttaattgattttaagatggaattttatattattaaataaataaattgcattaaatataagtaatttgtttccgtttaaagattagtaaaagggatcgtcgttcggrttgtatttactaaaatttcaaactaataaatatattatttaaatcctaaacatgctttcacaagtgaattatttatgattatttgttatattggtagtgtttgtgttacaaactattttaatgtaggatatagtattttttgtttaaggatgtttgagtatagtaaacaacagagaacaaaatagagaatgagagaagaataatgtaagtaattttaactttttttttttatgttgaattttgggtgttatttaagaatttgtttgtttttgtgggttttggatgaattaagtattgaaggaagaatggttaactatttgagagtttgagagttttggttattagttggtatttgtataattgaattgtggaataaataaataattatatttatgggtaatgtaattattaagtttttatatcttggttattggtgaaataaaaagaagaaagattgtttcgatgggtaattaaggtggagagagagagagttttattggaaagttaaaaagagaaaagaaactaataataatatttattattactaataatagtaaatatcattattgaataggtattctctcgttttacgtgcatcatcttctccaccacattcttcttccctcggaaaccctagcaaccgccaaggtttcgggcggtgcctccgtccgacagtcctccaactgaagccttgtcgccgtttgtcttccgtcgtcggtcgtcctcgcaaacatctggtctccttcaccatcgtcgtgaagcagccaaagcaagtcttcttcgttcgtccgaagccgaagcgtcacgaatcgcgcatcccaatcgtctgcgaccgccaactcgttcgcacatgtaggctttagtcttctgcaccgtgtttcttcacccgagccgtccgtgccatctctctacacatgagttcctcgcatcgactaccttcttagccgagtcgtgagccccttccttggttccttaaccatctttgctttggagccatatt

SEQ ID NO: 11 - DNA, 121 bp, Cucumis sativus
  source 1..121: mol_type = genomic DNA
aacctcgaaaggcaacactaacaaaaagaaatcaaacacttgaaatgcaaaatatggaaayaaaatagattgaaaaccctcaatactctcactagcaaagccaacaatcggagtactgctc

SEQ ID NO: 12 - DNA, 121 bp, Cucumis sativus
  source 1..121: mol_type = genomic DNA
tcgactcgaacctaaattcgctcactttctccgtcttcctaggcctgttaaggtaattttytactccaacctggattttatttctaagttttttctggctgaaaattnnnnntaannnnnn

SEQ ID NO: 13 - DNA, 1748 bp, Cucumis sativus
  source 1..1748: mol_type = genomic DNA
gtaataacccttttcctcttcctaatccatctcaggttcttcttcataatcctcctactactgctactacctccattatcgccgccgcttcttctcctatggacgattcctccgccactgcttggatcgacggtatcattaaggacttaatccatagctccactgccatatccatccctcagctcattcagaacgttcgtgagattatctacccgtgtaacccaaatcttgcgaatcttcttgagtttcgtcttcgtactttgacggaccctagtgttcctaacttcgctactgaggatcatcgagtgagaaagtcccctttgccgttgccggcgccggttgctggactggggttgcagcagaggcagtttaaccaagagcagcatgaacaagaacatgattgttctggactaaagcttaatctcgattctacttctcttcataatctttctaattttccctctcagccgccgtttcatgagccgtaccttcaatggggggcaacccctccgcctgtccccactccctccgccgctgccgccggcgaggatgccttacagcgcctccctggtcatcatcaacttaatctatcgtccgttacaccgtcgtcgcttgtttctttaaatcatgtcccttctaagccacaatcagaacagcagaactcttgtactaaggcggcggcggctgcacagccagctccagcaccaccatcgacgagcaataacccttcagcgactgctttgctgattagagagataaaagaggagatgagrcagcagaagagagacgaggaagggctacatctcttgactttgcttcttcaatgcgcagaagccgtttcagctgataatttagaagaagccaacaagatgctcttggaaatctccgagctatcgacaccgttcggcacatcggcccagagggtggcggcgtatttctctgaagcaatgtcggcgaggcttgtgagctcttgtttgggcatatatgcagctctgccgccgtcgttggtgccccatacacacagccagaaaatagcctcggccttccaaatcttcaatggcataagcccatttgtcaaattctcacatttcacagccaatcaagccatacaagaagcttttgaaagagaagagagagttcacatcatagatctagacatcatgcaaggacttcaatggcctggcctgttccacatcttggcgtctagacccggtgggccgccgtacgtccgccttacagggctgggcacctctcaggaagttcttgaagccactggcaaacgcctcactgaatttgctgagaagcttggccttccgtttgatttctttcccgtggctgataaaattggcaatctagacttggaaaggctcaacgtgagcaagagagaagccgttgctgttcattggatgcagcattctctttatgaagttactggttccgattccaatacgctatggcttttgcaaaggtaattggtaattcaactttcatatattcctggggtttcaccattaattaaattatttttggtaagtatttgttcatgatcaatactgattcttaatcttattcttaccttgtcgcttgtttaaaatgatgaatagttgttcagcacaccatataagtctataaaacaagtataaatttgcttagaaagacagctttcccttttttcatcttctctttcaaatctttcatgtgggtttggatcattgatatgtgggtttggaacattggtatgtgggttt